(19) United States
(12) Patent Application Publication
(10) Pub. No.: US 2025/0352410 A1
(43) Pub. Date: Nov. 20, 2025

(54) ABSORBENT SYSTEMS AND ABSORBENT ARTICLES INCLUDING THE SAME

(71) Applicant: Coterie Baby, Inc., New York, NY (US)

(72) Inventor: Xiaoqing Liu, Xiqiao Town (CN)

(21) Appl. No.: 19/005,038

(22) Filed: Dec. 30, 2024

(30) Foreign Application Priority Data
May 17, 2024 (CN) 202421088007.9

(51) Int. Cl. A61F 13/534; A61F 13/15; A61F 13/51; A61F 13/53

(52) U.S. Cl. CPC A61F 13/53418; A61F 13/15203; A61F 13/51; A61F 2013/15463; A61F 2013/530131; A61F 2013/53051; A61F 2013/53062; A61F 2013/530737; A61F 2013/530868

(57) ABSTRACT

An absorbent system including a first absorbent core layer, the first absorbent core layer made up of cellulose and superabsorbent material, a second absorbent core layer disposed above the first absorbent core layer, the second absorbent core layer made up of synthetic material and superabsorbent material, the second absorbent layer having end portions that are folded so that the second absorbent layer has a C-shape, a first acquisition-distribution layer disposed over the second absorbent core layer, a core wrap disposed around the first absorbent core layer, the second absorbent core layer, and the first acquisition-distribution layer, and a second acquisition-distribution layer disposed over the core wrap.

### FIELD OF THE INVENTION

The present invention generally relates to absorbent systems, and in particular to absorbent systems useful in hygienic absorbent articles.

### BACKGROUND

Absorbent articles, such as, for example, diapers, training pants, briefs, sanitary pads and pantiliners, have been made and sold for many years. In general, these products prevent accidental contact with body fluids or feces. The products typically include an absorbent core to retain fluids.

Conventional absorbent cores may be “pre-made” of synthetic materials. The term “pre-made” in this context means that the core is made of synthetic materials and affixed to the absorbent article during the manufacturing process.

One well known problem associated with premade cores using only synthetic carriers with embedded super-absorbent particles (SAP), has always been related to the slow intake speed of liquids. To make matters worse, premade cores with channels perform poorly when the diaper is at an angle, as the liquid can quickly run out inside the channel due to gravity. This can be explained by the lack of cellulose fibers that can help with the wicking and act as a temporary reservoir giving time for the SAP to do its job. Even with traditional diaper cores, like those using a blend of cellulose and SAP, when the ratio of SAP is increased, for example above 50%, typically it takes longer for fluids to be absorbed as compared with a core with a lower SAP ratio. Increasing the amount of SAP higher than 50% SAP ratio is important while designing a diaper for extended use, for example those expected to last the whole night. This is because SAP can hold a much higher amount of fluid, like urine, as compared to cellulose, especially under pressure. Additionally, the higher SAP ratio helps to avoid a bulky core that may not be comfortable due to its excessive thickness, and in some cases may even interfere with the normal walking of a baby, particularly after the core has been saturated with urine.

In an effort to reduce early leakages, cores loaded with higher SAP ratios need a special layer of nonwoven material, referred to in the industry as an ADL, or Acquisition Distribution Layer. The ADL is typically placed between the absorbent core and the topsheet. The ADL is typically made of high loft polyester, typically from 30 GSM to 120 GSM. A more resilient ADL material is typically preferred to help with liquid distribution and to keep the top surface as dry as possible. For a high-performance absorbent core, it is extremely important to avoid having a pool of liquids inside the diaper at any time, as this can lead to early leakages, even when the size of the urine is well below the expected retentive capacity of the product. The ADL helps with the intake of fluids and also with wicking to take better advantage of the absorbent materials in the core, thereby helping to avoid the formation of a pool of liquid.

If the ratio of SAP is increased in an absorbent core, a larger weight ADL (i.e., higher basis weight) is needed to compensate for the slower speed. For example, if a diaper core with a ratio of SAP, for example of more than 60% by weight, does not use an ADL, it will fail terribly due to premature leakages. On the other hand, if a diaper with a minimal amount of SAP, for example, a diaper core with less than 10% SAP ratio is used with an ADL (independently of using an even higher basis weight), the ADL would be a complete waste of resources, as it does not add value to an already fast core. But in this case the core will have a very limited retentive capacity and no benefit in terms of rewet. An effective balance between SAP ratio and ADL basis weight, particularly in the case of nonwoven ADLs, is needed to reach optimal performance, avoiding adding resources that no longer add value.

As a way to reduce the acquisition time of fluids, Procter & Gamble (“P&G”) recently developed a SAP printing technology that allows for production of an absorbent core with “channels.” These channels are not made full length, along the length of the core, but instead are interrupted at both ends of the core for about one third of its length, to avoid the possibility of liquid running outside the channel, and then leaking prematurely out of the diaper. This is a problem that can easily be shown when the diaper is at an inclined angle and uses a full-length channel (a channel with no interruptions). Once the SAP starts to absorb liquids, it swells, creating the channels. P&G was among the first to make a fluff-less core in the western world, except for the small amount of curly fiber and the ADL used on top of the printed SAP. Other manufacturers, such as Drylock Technologies, also developed a fluff-less core made in the shape of small pillows, bonded with ultrasound, but soon it was replaced with their own alternative for channels, but using only a small amount of fluff. Ontex, Abena and Kimberly-Clark soon developed their own alternative versions of interrupted channels, each with a different configuration.

Interrupted channels reduce the acquisition time by moving the liquids quickly along the channel, particularly after the initial swelling of the SAP. Many manufacturers had to fine tune their own channels, for example, by changing the length and the distance to the edges for channel interruption, and at the same time they were learning how to reduce early leakages.

As more companies tried to use the concept of channels, they were confronted by the same problem they initially had back in time when they used three-dimensional drum former pockets to produce 3D absorbent cores. A diaper core with channels has to be registered, meaning that each time the diaper machine has to be changed to a different diaper size, the absorbent core has to be changed too. This creates not only increased down time, but also brings more adjustments and overall complexity to the production downstream, increasing waste and reducing overall efficiencies.

There is an ongoing challenge in making a high-performance absorbent article in that key properties of the article need to be balanced to achieve a comfortable experience for the wearer, while at the same time providing a longer lasting product without risk of preliminary leakages. There is also an ongoing challenge in making diaper absorbent cores with channels in terms of reducing downtime and waste while increasing overall manufacturing efficiencies.

### SUMMARY OF THE INVENTION

An absorbent system according to an exemplary embodiment of the present invention comprises: a first absorbent core layer, the first absorbent core layer comprising cellulose and superabsorbent material; a second absorbent core layer disposed above the first absorbent core layer, the second absorbent core layer comprising synthetic material and superabsorbent material, the second absorbent layer having end portions that are folded so that the second absorbent layer has a C-shape; a first acquisition-distribution layer disposed over the second absorbent core layer; a core wrap disposed around the first absorbent core layer, the second absorbent core layer, and the first acquisition-distribution layer; and a second acquisition-distribution layer disposed over the core wrap.

In an exemplary embodiment, the second absorbent core layer comprises a nonwoven layer disposed between two carrier layers.

In an exemplary embodiment, the carrier layers comprise spunbond material.

In an exemplary embodiment, the second absorbent core layer comprises a channel formed between the end portions.

In an exemplary embodiment, the first absorbent core layer comprises a channel.

In an exemplary embodiment, the first absorbent core layer comprises a first portion and a second portion, and the channel of the first absorbent core layer is formed by a space between the first portion and the second portion.

In an exemplary embodiment, the channel of the first absorbent core layer is formed by a portion of the second absorbent core layer that has a reduced amount of superabsorbent material.

In an exemplary embodiment, the channel of the second absorbent core layer is formed by a portion of the second absorbent core layer that has reduced thickness.

In an exemplary embodiment, the first acquisition-distribution layer is larger than the second acquisition-distribution layer.

In an exemplary embodiment, the second acquisition-distribution layer extends less than a full length of the absorbent system.

An absorbent article according to an exemplary embodiment of the present invention comprises: an absorbent system according to an exemplary embodiment of the present invention comprises: a first absorbent core layer, the first absorbent core layer comprising cellulose and superabsorbent material; a second absorbent core layer disposed above the first absorbent core layer, the second absorbent core layer comprising synthetic material and superabsorbent material, the second absorbent layer having end portions that are folded so that the second absorbent layer has a C-shape; a first acquisition-distribution layer disposed over the second absorbent core layer; a core wrap disposed around the first absorbent core layer, the second absorbent core layer, and the first acquisition-distribution layer; and a second acquisition-distribution layer disposed over the core wrap.

### BRIEF DESCRIPTION OF THE DRAWINGS

FIG. 1A is a cross-section view of an absorbent system according to an exemplary embodiment of the present invention;

FIG. 1B is a cross-section view of a top absorbent core layer according to an exemplary embodiment of the present invention;

FIG. 2A is a cross-section view of a top absorbent core layer according to an exemplary embodiment of the present invention; and

FIG. 2B is a plan view of a top absorbent core layer according to an exemplary embodiment of the present invention.

### DETAILED DESCRIPTION OF PREFERRED EMBODIMENTS

Absorbent systems according to exemplary embodiments of the present invention include a primary core layer and a C-fold synthetic core layer disposed on top of the primary core layer. This unique construction provides enhanced performance parameters as compared to conventional cores, including lower leak rates, lower rewet in long-term scenarios and high saline retention, as well as optimized combinations of these parameters.

As used herein, the term “absorbent core” refers to a material or combination of materials suitable for absorbing, distributing, and storing aqueous fluids such as urine, blood, menses, and water found in body exudates. Absorbent cores or inserts may be formed or cut out from rolls of absorbent materials. The size and shape of the absorbent core can be altered to meet absorbent capacity requirements, and to provide comfort to the wearer/user. The length of the absorbent core may range from about 20 cm to about 60 cm. The width of the absorbent core may range from about 4 cm to about 20 cm. When a second absorbent core is utilized, the second absorbent core may be the same size as the first core, smaller than the first core, or larger than the first core. Nonlimiting examples of liquid absorbent materials suitable for use as absorbent cores in accordance with exemplary embodiments of the present invention include comminuted wood pulp, which is generally referred to as airfelt; creped cellulose wadding; absorbent gelling materials including superabsorbent polymers, such as hydrogel-forming polymeric gelling agents; chemically stiffened, modified, or cross-linked cellulose fibers; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof, as is well known in the art of making absorbent products such as sanitary napkins, pantiliners, incontinence pads, and the like. The amount of superabsorbent polymer in the absorbent core may range from about 20 to about 85 or about 40 to about 80 or about 50 to about 75 or about 60 to about 70 percent by weight, based on the total weight of absorbent material in the core.

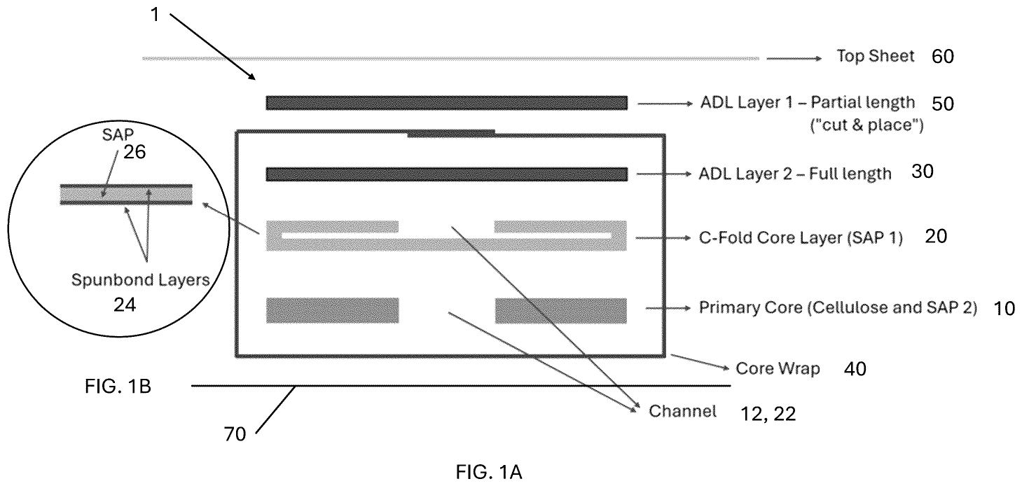

FIG. 1A shows an absorbent system, generally designated by reference number 1, according to an exemplary embodiment of the present invention. The absorbent system 1 includes a first absorbent core layer 10 (also referred to herein as a “bottom absorbent core layer”), a second absorbent core layer 20 (also referred to herein as a “top absorbent core layer”) disposed above the first core absorbent layer 10, a first ADL layer 30 disposed above the second absorbent core layer 20, a core wrap 40 disposed around the first absorbent core layer 10, the second absorbent core layer 20, and the first ADL layer 30, and a second ADL layer 50 disposed above the core warp 40 and the first ADL layer 30. One or more of the layers may be attached to one another by, for example, adhesive.

The first absorbent core layer 10 is a homogeneously blended absorbent core, made with SAP and cellulose. The first absorbent core layer 10 may be a unitary sheet or be made of separate sheets that are arranged side by side. In an exemplary embodiment, the first absorbent core layer 10 includes a middle channel 12 formed by, for example, an opening formed partially or completely through the first core layer 10, a separation between two sheets of material arranged side by side that make up the first absorbent core layer 10, and/or an area of the first absorbent core layer 10 that is devoid of or contains a lesser amount of SAP. When the first absorbent core layer 10 swells from fluid absorption, the channel 12 is surrounded by material that is more swollen as compared to the channel 12, which enhances the fluid absorption properties so that speed of acquisition is further improved. In exemplary embodiments, the middle channel 12 may extend the full length or partially along the length of the first absorbent core layer 10.

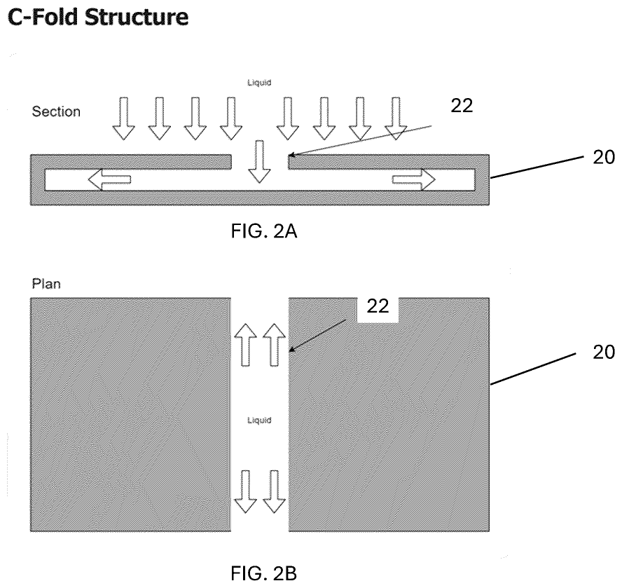

As shown in FIG. 1B, the second absorbent core layer 20 is a pre-made synthetic absorbent core made up of two carrier layers 24 and a nonwoven layer 26 disposed between the carrier layers 24. In an exemplary embodiment, the carrier layers 24 are made of spunbond material and the nonwoven layer 26 is a high loft polyester nonwoven embedded with low-speed, high permeability SAP. As shown in FIGS. 2A and 2B, the lateral end portions of the second absorbent core layer 20 are folded over the top of the second absorbent core layer 20 so that the second absorbent core layer 20 takes on a “C-shape”. This folding creates a central channel 22 in the middle of the absorbent article running the full length of the second core layer 20. The central channel 22 created with absorbent material helps to avoid run-offs, which is a typical problem of absorbent articles with a full-length channel. In this regard, the arrows in FIGS. 2A and 2B show the direction of liquid flow through the second absorbent core layer 20.

The first and second ADL layers 30, 50 may be made entirely of conventional fibrous materials with little absorbency, but in some embodiments includes water-absorbent polymer particles or other absorbent materials. The fibrous material may be hydrophilic, hydrophobic or can be a combination of both hydrophilic and hydrophobic fibers. The fibrous material may be derived from natural fibers, synthetic fibers or a combination of both. Suitable ADLs are formed from cellulosic fibers and/or modified cellulosic fibers and/or synthetics or combinations thereof. Thus, suitable ADLs may contain cellulosic fibers, in particular wood pulp fluff. Modified cellulosic fibers may be utilized for fluid acquisition and distribution. Examples of modified cellulosic fibers are chemically treated cellulosic fibers, especially chemically stiffened cellulosic fibers. The basis weight of cellulosic fibers and modified cellulosic fibers may range from about 50 to about 200 gsm.

Suitable ADL layers may further include synthetic fibers. Hydrophilic synthetic fibers may be obtained by chemical modification of hydrophobic fibers, such as by surfactant treatment of hydrophobic fibers. The surface of the hydrophobic fiber can be rendered hydrophilic by treatment with a nonionic or ionic surfactant, e.g., by spraying the fiber with a surfactant or by dipping the fiber into a surfactant.

In some embodiments, the ADL layers 30, 50 comprise fibrous material and water-absorbent polymer particles distributed within to function as an absorbent layer. ADLs may include from about 80% to about 100% by weight fibrous material and from 0% to about 20% or about 5% to about 15% or about 10% by weight water-absorbent polymer particles, based on the total weight of the ADL.

Alternatively, a bundle of synthetic fibers acting as an ADL loosely distributed on top of the fluid-absorbent cores may be used. Suitable synthetic fibers include, for example, copolyester, polyamide, copolyamide, polylactic acid, polypropylene or polyethylene, viscose or blends thereof. Bicomponent fibers may also be used. In exemplary embodiments, the synthetic fiber component may be composed of either a single fiber type with a circular cross-section or a blend of two fiber types with different cross-sectional shapes.

The ADL basis weight may range from about 20 gsm to about 200 gsm, depending on the concentration of water-absorbent polymer particles. The length of upper ADLs may range from about 6 cm to about 25 cm. The width of upper ADLs may range from 4 cm to 12 cm. The length of lower ADLs may range from about 6 cm to about 60 cm. The width of lower ADLs may range from 6 cm to 15 cm.

In exemplary embodiments, the core wrap 40 may assist with containment and integrity of the absorbent core components. The core wrap 40 may be bonded to one or more both of the first and second core layers 10, 20. Bonding of the core wrap 40 to the absorbent cores 10, 20 may occur via any means known to one of ordinary skill, such as, but not limited to, adhesives, such as, for example, hot melt adhesives in the form of spray, melt-blown, multi-lines, slot, etc. The core wrap 40 may be composed of separate sheets of material (such as an upper core wrap and a lower core wrap) which can be utilized to partially or fully encompass the absorbent cores 10, 20 and which can be sealed together using a sealing means such as an ultrasonic bonder or other thermochemical bonding means, or the use of an adhesive. Alternatively, as shown in FIG. 1A, the core wrap 40 may be composed of only a single sheet of material that is wrapped around the cores 10, 20. The core wrap 40 may include, but is not limited to, natural and synthetic fibers such as polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers may include wool, cotton, flax, hemp, and wood pulp. The material forming the core wrap 40 may be selected from meltblown-spunbond-meltblown fabric, spunbond fabric, meltblown fabric, coform fabric, carded web, bonded-carded web, bicomponent spunbond fabric, spunlace, tissue, and combinations thereof. Further, the core wrap 40 may be made of a spunbond-meltblown-spunbond (“SMS”) material, such as a 9 gsm spunbond-meltblown-spunbond material.

The core wrap 40 may be less hydrophilic than the absorbent cores 10, 20, but sufficiently porous to permit body fluids to penetrate through the core wrap 40 to reach the absorbent cores 10, 20. The core wrap 40 may have sufficient structural integrity to withstand its own wetting and the wetting of the absorbent cores 10, 20. In order to support this functional property of the core wrap 40, a wet strength agent may be applied to the core wrap 40. A non-limiting example of a wet strength agent may be Kymene 6500 (557LK) or equivalent available from Ashland Inc. of Ashland, Ky., U.S.A. Similarly, a surfactant may be included in the core wrap 40 to promote hydrophilicity.

The combination of the first and second absorbent core layers 10, 20 provides a solution to the problems associated with using either traditional cores or synthetic cores alone, particularly when the second absorbent core layer 20 (which is a pre-made, synthetic core) is made as described using a folding on both edges that create a central channel. By carefully selecting SAPs with different properties, for example a SAP with slow speed and high permeability for the premade core 20 at the top, and a SAP with faster speed at the bottom in the traditional core 10, it is possible to quickly accept the intake of fluids through the central channel without the risk of having any runoffs, even when the diaper is inclined. This is because the lower floor of the channel 22 has absorbent material that slows the speed of the runoff, but also at the same time allows liquids to go through deeper into the traditional core 10 below where high speed SAP is present.

Additionally, by carefully selecting the ADLs used in this new core construction, it is possible to optimize liquid intake. This way it is possible to use ADLs with different densities, creating an effective density gradient that helps move liquids faster while making it difficult for liquids to come back. In this regard, in exemplary embodiments, the first ADL layer 30, which is disposed above the premade core 20, may be a full-length ADL, while the second ADL layer 50, which is disposed above the core wrap 40, may be a reduced length ADL (e.g., an ADL patch). Without being bound by theory, this construction takes advantage of the fact that liquids typically travel from open structures to more dense structures, resulting in a check valve analogy. It is advantageous to use a reduced length ADL layer at the top of the core wrap 40 to reduce potential leakages at the top and bottom of the absorbent core edges and reduce additional costs by locating the reduced length ADL layer just on the target zone. In exemplary embodiments, the second ADL layer 50 may not have the same basis weight as the first ADL layer 30.

The absorbent system 1 may be useful for, for example, diapers, training pants, youth pants, briefs, sanitary pads, bladder control pads and the like. In use, the absorbent systems of exemplary embodiments of the present invention are placed on a top surface of a backsheet (for example, backsheet 70 shown in FIG. 1A). Backsheets are materials that generally are liquid impermeable but may be moisture vapor permeable (breathable). Backsheets are used in absorbent products on a surface of the product that is distal to the user's body. The backsheet can be made of any known or otherwise effective backsheet material, provided that the backsheet prevents external leakage of exudates absorbed and contained in the protective underwear. Flexible materials suitable for use as the backsheet include, but are not limited to, woven and nonwoven materials, laminated tissue, polymeric films such as thermoplastic films of polyethylene and/or polypropylene, microporous films, composite materials such as a film-coated nonwoven material, or combinations thereof, as is well known in the art of making absorbent products, such as sanitary napkins, pantiliners, incontinence pads, and the like. The water vapor transmission rate of a breathable backsheet may be, for example, in the range of 0 to 9,000 g/m² per 24 hours.

The absorbent system is typically attached to the backsheet with an adhesive. Suitable adhesives are known in the art and include hot melt adhesives, emulsion polymer adhesives and the like.

A topsheet or cover (such as topsheet 60 shown in FIG. 1A) is placed on top of the absorbent system and attached to the absorbent system and backsheet with adhesive, ultrasonic bonding or combinations thereof, forming a chassis. Suitable topsheets are compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. A suitable topsheet can be made of various materials, such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making absorbent products such as sanitary napkins, pantiliners, incontinence pads, protective underwear and the like.

Elastic side panels may be attached to the chassis to form diapers or adult protective underwear. Any elastic side panel known in the art of absorbent articles may be useful. Suitable elastic side panels include laminates of elastic films with nonwovens, laminates of elastic strands with nonwovens and the like. The elastic panels may be attached to the chassis by adhesive, ultrasonic bonding or a combination thereof. The length, width and shape of the side panels may be designed to make products of different sizes. Products with side panels may have a more underwear like appearance. A portion of each side panel is left unattached to form leg openings. The side panels may be attached to the chassis at various angles to create a more garment like fit.

As is known in the art, hooks and loops may be used on articles in accordance with exemplary embodiments of the present invention. Nonwoven materials may function as the loops.

In exemplary embodiments, the hook fasteners may be made up of separate hook elements or may be integral with the side panels. In this regard, the hook elements may be bonded to the side panels by adhesive, ultrasonic, thermal bonding or the like. Alternatively, the hook elements may be intimately joined with the material that forms the side panels. The hook elements may be arranged on the side panels in longitudinally extending strips that are laterally spaced from one another. Alternatively, the hook elements may be arranged in a pattern of geometric shapes or lines. Desirably, the hook elements are arranged on an inelastic material in order to improve ease of processing and the shear strength of the seam.

Table 1 below provides materials, material names, material compositions, basis weight ranges, and preferred basis weights for components of an absorbent article according to an exemplary embodiment of the present invention. The materials and material properties used in exemplary embodiments of the present invention are not limited to those provided in Table 1. For example, nonwoven material made of fibers other than those listed in Table 1 may be used, such as, for example, bicomponent polypropylene-polyethylene, polyester, and polybutylene terephthalate, to name a few.

TABLE 1

| | | | | BASIS | BASIS |
|---|---|---|---|---|---|
| | | | | WEIGHT | WEIGHT - |
| | | MATERIAL | MATERIAL | RANGE | PREFERRED |
| COMPONENT | MATERIAL | NAME | COMPOSITION | (GSM) | (GSM) |
| Topsheet | Nonwoven | Pearl Dot Hot Air | Polyethylene, | 10 to 35 | 25 |
| | Fabric | Nonwoven Fabric | Polybutylene | | |
| | | | Terephthalate | | |
| Leg Cuffs | Nonwoven | SMS Soft | Polypropylene | 6 to 25 | 13 |
| | Fabric | Hydrophobic | | | |
| | | Nonwoven Fabric | | | |
| Core Wrap | Nonwoven | SMS Hydrophilic | Polypropylene | 6 to 20 | 10 |
| | Fabric | Nonwoven Fabric | | | |
| Acquisition Layer - | ADL patch | Acquisition | Polyethylene, | | |
| TOP | (placed on top | Distribution Layer | Polybutylene | | |
| | of premade | (ADL) | Terephthalate | | |
| | core) | | | | |
| Core - TOP | Premade Core | Box-style | Nonwoven Fabric, | 150 to 500 | 434 |
| | (used on top of | Composite Paper - | Acrylic acid, | | |
| | traditional core) | C Fold Core | Adhesive, | | |
| | | | Polyethylene, | | |
| | | | Polybutylene | | |
| | | | Terephthalate | | |
| Carrier Layer - | Nonwoven | SMS Hydrophilic | Polypropylene (PP) | 6 to 25 | 21 |
| TOP | Fabric | Nonwoven Fabric | | | |
| Nonwoven Fabric | High loft | Fluffy Nonwoven | Polypropylene (PP) | 25 to 60 | 45 |
| (between carrier | Nonwoven | Fabric | | | |
| layers) | | | | | |
| Carrier Layer - | Nonwoven | SMS Hydrophilic | Polypropylene (PP) | 6 to 25 | 18 |
| BOTTOM | Fabric | Nonwoven Fabric | | | |
| SAP - Top Core | SAP | Sandia China SAP | SODIUM | 50 to 200 | 145 |
| | | 930NP | POLYACRYLATE | | |
| Adhesive (used to | Hot melt | 2885U | Polymers, plasticisers, | 4 to 15 | 7 |
| bond premade | | | resins, and oils | | |
| core) | | | | | |
| Acquisition Layer - | ADL full length | ADL | Polybutylene | 25 to 60 | 52 |
| BOTTOM | | (100% 12D) | Terephthalate | | |
| SAP - BOTTOM | SAP | SUMITOMO | SODIUM | 50 to 500 | 150 |
| Core | | JAPAN SAP | POLYACRYLATE | | |
| | | (SA60s) | | | |
| Pulp - BOTTOM | Pulp | Pulp | Cellulose Fiber | 20 to 200 | 100 |
| Core | | (416/411/DT) | | | |
| Adhesive (used to | Hot melt | DMC2888U | Polymers, plasticizers, | N/A | |
| bond ADL) | | | resins, and oils | | |
| Adhesive | Structural Glue | Structural Glue | Polymers, plasticizers, | N/A | |
| (Construction) | | | resins, and oils | | |
| Adhesive (End | End Seal Glue | End Seal Glue | Polymers, plasticizers, | N/A | |
| Seal) | | | resins, and oils | | |

Since the launch of the very first diaper, three properties have always remained critical. These properties are the retentive capacity, the speed of acquisition, and the rewet (collectively known as “Tripod Properties”).

The retentive capacity measures the amount of urine that a diaper can hold under a specific pressure. For diapers that use a mostly flat core along its width and length, retentive capacity can be measured directly by applying different pressures on top of the wet core, for example using a plate that copies the shape of the core and a pneumatic piston to add pressure. For situations where the absorbent core is not flat, for example when testing diapers using three dimensional absorbent cores, a centrifugal machine can be used to indirectly correlate its retentive capacity. The retentive capacity, assuming a diaper will not show preliminary leakages associated to a slow intake of fluid with respect to the speed of the insult, correlates well with the duration that a diaper can be used under typical situations before it will start to leak, for example before it reaches its design capacity. Ideally, the retentive capacity is relatively high to allow a diaper to last longer so that less diapers are needed per day. The following provides a method for measuring retentive capacity using pressure (i.e., “Free Swell Capacity”), and another method using a centrifugal machine (i.e., “Retention Capacity”).

### Retentive Capacity Test Method

This method describes the procedure for the determination the absorption and retention of 0.9% saline in infant absorbent hygiene products (AHP's).

Infant AHP's have a variety of absorbent materials and absorbent core designs with varying amounts of fibers and absorbent polymers. The amounts of these materials are set to offer specific total capacity and retention when used by consumers. Often, the targets for these two values are the result of specifically designing the AHP for the intended use: for example, infants of a specific age or size, day or night use, premium and value AHP's. This test allows a direct comparison of these types of products which can aid in understanding the consumer use expectations.

In this test, AHP's fully absorb saline and then release free fluid by gravity to give the freeswell capacity. Centrifugation removes interstitial fluid to give the retention capacity.

Reagents: 1. 0.9% w/w sodium chloride (NaCl) solution made from solid NaCl and distilled or deionized water (see work instruction WI 001 for making saline solution).

Equipment and Materials: Digital Timer; Scissors; Digital Scale capable of reading to 0.1 g with a 1000 g or higher capacity; Centrifugal dryer, 3200 rpm with 23 inch ID drum (Panda PANSP23B or equivalent); Submersion tray capable of holding 20 liters of 0.9% saline and of dimensions to hold six AHP's; Fixed rack above a sink to hold AHP's while gravity draining; Clamps; Large bowl.

Conditions: AHP's were removed from packaging and allowed to equilibrate at ambient conditions for 15 minutes (this allows the AHP's to relax from compression packaging).

The ambient lab and testing conditions were, preferably, 23±1° C. with RH of 50±2%. If these conditions could not be maintained, the actual test conditions were recorded and included in the report.

General Operation: The elastics that keep the AHP in a cupped position were cut to allow the AHP to lie flat. The AHP was immersed in 0.9% saline for a specific amount of time and then removed and allowed to drain. This provided the absorption capacity. The product was then centrifuged to determine the retention capacity.

Procedure:

1. All of the diapers from a bag (or multiple bags) of the AHP being tested were weighed. The average diaper weight for all of the AHP samples were weighed. Each of the AHP's were marked with identifying text and numbers. Six (6) AHP's near the average weight were selected; the ID and weight of each product were recorded as WDN, where N is the sample number from 1-6.

2. The first AHP was selected and the outer leg gathers of both sides of the product were carefully cut at several places, taking care to cut through the elastics but not through to the absorbent core.

3. Next, the inner leg gathers (cuffs) on each side were cut at several places taking care to cut the elastics but not cut into the absorbent core. All of the cuts will allow the AHP to remain flat during submersion.

4. Steps 2 and 3 were repeated for the other 5 samples.

5. The submersion tray was filled with 20 liters of 0.9% saline.

6. The timer was reset to zero and the AHP's were stacked in numerical order near the submersion tray.

7. The first sample was held at both ends so that it was open and flat with the porous top sheet facing down, toward the saline. The sample was placed in the submersion tray and the timer was started. The sample was gently pressed into the saline so that it floated but was not submerged.

8. When the timer reached 10 seconds, the second sample was submerged in the same way as step 7. This action was repeated every 10 seconds until all samples were submerged, making sure that they did not overlap.

9. When the timer reached 10:00 minutes, the first AHP was removed and hung on the bar on the fixed rack over the sink using clips. The porous top sheet was faced forward. Care was taken not to inadvertently squeeze fluid from the AHP.

10. Step 9 was repeated for the other 5 AHPs at 10-second intervals.

11. When all samples were hung, the lid to the submersion tray was quickly placed lengthwise across the width of the tray. This is where the samples will be placed for transport and subsequent operation.

12. When the timer reaches 12:00 minutes, the first AHP was removed, gently folded and laid on the submersion tray lid.

13. Step 12 was repeated at 10-second intervals for the remaining 5 AHP's.

14. The large bowl was placed on the scale and the zero (or tare) button was pressed so that 0.00 g was in the display.

15. Weigh each AHP was weighed and the weight was recorded as WFSN, where N was the AHP number from 1 to 6 (FS stands for “freeswell”). The scale was re-zeroed/re-tared between each weighing.

16. The centrifuge was plugged in and a container was positioned below the drain to capture the fluid that was removed from the AHP's.

17. The first samples was placed in the centrifuge with the porous top sheet facing outward against the drum and snugly following the curvature of the drum. [NOTE: The absorbent cores of some AHP's can sag during the hanging/draining operations of steps 9-12. If this occurred, the AHP was gently pressed so that the absorbent core was distributed evenly in the AHP. This improved the balance of the AHP in the centrifuge and assured even dewatering.]

18. The second sample was placed into the centrifuge in the same way as the first sample but on the opposite side of the drum. This balanced the load during centrifugation.

19. The centrifuge lid was closed. The handle was turned to the right (clockwise) to turn on the centrifuge and, simultaneously start the timer. The centrifuge was ran for 1 minute and 6 seconds (the 6 seconds accounts for the time required for the centrifuge to come up to 3200 rpm).

20. The handle was turned to the left (counterclockwise) to stop the centrifuge. The centrifuge was allowed to gradually come to a stop on its own.

21. Assurance was made that the bowl was on the scale and that the scale had been zeroed/tared so that the screen read 0.00 g.

22. The first sample was weighed, and the weight was recorded as WRN, where N was the AHP number (R stands for “retention”). The scale was re-zeroed and this process was repeated for the second sample.

23. Steps 16-22 were repeated for the remaining AHP's pairwise.

24. If further tests were to be carried out, the submersion tray was refilled with saline to 20 liters; otherwise, the submersion tray was emptied and rinsed out.

25. To prevent corrosion in the centrifuge, at the end of all testing, the drum of the centrifuge was rinsed with 1-2 liters tap water and the centrifuge was ran to send the water through the drain lines. The centrifuge drain container was emptied in the sink.

Calculations:

$$Freeswell Capacity ( FSC ) = WFSN - WDN$$

$$Retention Capacity ( CRC ) = WRN - WDN$$

Speed of acquisition attempts to measure the time it takes for an insult of urine to be absorbed by the core. A timer is started once the insult is added to a cylinder placed in direct contact with the absorbent core, and stopped once there is no more visible liquid in the cylinder. There are many methods to measure this, for example measuring the time it takes for an amount of insult to be fully absorbed using different diameter cylinders; or using funnels with different angles and different diameter orifices, among many others. The acquisition time correlates well with potential preliminary leakages. If it is too slow, it may allow the formation of liquid pools of urine that have not yet been absorbed by the core. That pool of liquid, under certain special conditions, may end in preliminary leakages, leakages that occurred before the diaper reached its retentive design capacity, such as, for example, while the diaper is at an angle and the diaper has only absorbed a small percentage of its design capacity. Many sealing features have been added to diapers, like leg gathers, leg cuffs, waist elastic, etc. Most times these features can reduce some of the leakages, but most times are unable to stop all leaks when the acquisition speed is too slow. The following is a description of the test method used to obtain acquisition time data provided in this application.

### Acquistion Time Test Method

The following test was performed to determine the acquisition time of a diaper.

The tests were performed with a four-time urine substitute liquid addition. The amount of liquid applied depended on the size of the diaper, as follows: Newborn (4×30 ml); Mini (4×40 ml); Midi (4×50 ml); Maxi/Junior (4×70 ml). Five (5) samples of each product were tested. The testing equipment included: foam plastic mat covered with hook material; 8 kg weight with funnel on top (base area 100 mm×300 mm; ca. 0.4 psi); electronic time clock (accuracy 1 s per 20 min); scale (accuracy±0.01 g); filter paper according to testing laboratory Hy-Tec Hygiene Technology GmbH (100 mm×300 mm; Schleicher & Schuell type 604); beaker. At least five unused diapers were tested. The dry weight of each sample was determined and noted down. The insult point was marked on the diaper according to the gender-specific position, i.e. in the center of the total diaper for girl, 2.5 cm towards the front for unisex and 5 cm towards the front for boy. Afterwards the filter paper was positioned with its center on the insult point. The diaper was mounted on the foam plastic mat. The 8 kg weight was placed on the test devices so that the mark on the dosing point was in line with the marking on the diaper. The 0.9% NaCl solution was poured onto the diaper through the tube. The time the liquid needed for penetrating the topsheet was measured and recorded. Once the liquid was absorbed by the diaper a time clock was started to measure a waiting period. After a waiting period of 5 minutes the same amount of liquid was poured onto the diaper for the second time and the absorption time was measured again and recorded. This procedure was repeated four times in total. After the fourth waiting period of 5 minutes the weight was taken off the diaper and any liquid remaining on the base plate was wiped off. Of the tested products, the average and standard deviation of the absorption time (in seconds) after fourth liquid addition was recorded as the average acquisition time for all 4 insult times. After the last addition and a waiting period of 5 minutes a rewet measurement with the same test device and the above mentioned load was ran for 15 s with 20 pieces of the above mentioned filter paper. The release of liquid was recorded and reported as average of 5 (five) single samples.

Rewet attempts to measure users' comfort. If the skin of the baby is wet while the baby is using the diaper, it will soon feel cold due to the room temperature typically being colder than the body temperature. This is amplified by use of modern breathable diapers that allow for liquid evaporation and will result in an even colder feeling. The wetter the skin the more uncomfortable for the user, oftentimes resulting in a baby crying or an adult changing to another brand. Rewet measures the amount of liquid that can return to the surface using a filter paper under pressure. It attempts to measure skin comfort. The drier the skin, the more comfortable it will be. To measure rewet, first an insult is added into the core, then allowed to be absorbed for a specific amount of time, and then filter papers are placed on top of the core under a pressure to measure the amount of liquid that returns to the surface. The following is a description of the test method used to obtain rewet data provided in this application.

### Rewet Test Method

The following test was performed to determine the rewet of a diaper under day and night conditions.

The test was carried out with two liquid intakes of urine solution and/or three liquid intakes of urine solution. The amount of liquid applied depended on the size of the diaper as follows: Newborn (3×30 ml//70 ml/30 ml); Mini (3×40 ml//80 ml/40 ml); Midi (3×50 ml//100 ml/50 ml); Maxi/Junior (3×70 ml//150 ml/50 ml). A 20 minute waiting period was taken between the start of each intake and the measurement to allow liquid distribution and storage in the diaper. The testing equipment included: 4 kg weight; balance with 0.01 g accuracy; Plexiglas funnel 14×19 cm; filter paper stack (Type 604) 6-12 layers at 140×190 mm; filter paper 14×19 cm; test liquid (0.9% NaCl-solution in deionized water); Plexiglas table; brackets; stopwatch; dosing table (2 ml/s flow rate); Optifix Dispenser 70 ml+50 ml. Five (5) samples of each product were tested. The sample was weighed and placed on the table with clips for flat wrinkle-free positioning. The dosing table was placed above the diaper core (Girl diapers in the middle, unisex diapers 2.5 cm forward to belly end, boy diapers 5 cm forward to belly end). Liquid quantity in the dosing table was measured, depending on the test samples size. The stopwatch was started at the beginning of fluid intake. 6-12 sheets of filter paper were weighted and recorded as dry weight. After expiry of 20 minutes, the filter paper (according to testing laboratory Hy-Tec Hygiene Technologie GmbH (190 mm×140 mm; Schleicher & Schuell type 604)) was centered on the liquid inlet point, then plexiglass plate was placed on the diaper and loaded with 4 kg for 15 seconds. The filter paper was then weighed. Difference between the filter paper weight before and after the test was the rewet. For the second application of fluid, the process is repeated starting from the measurement of liquid quantity in the dosing table. The product weights were recorded to an accuracy of 0.1 g and the remaining weights to 0.01 g.

The following provides a method of measuring incline run off.

### Incline Runoff Test Method

1. 0.9% sodium chloride solution was prepared (using deionized or distilled water or equivalent and ACS grade or better NaCl), using coloring to allow visualization of fluid trajectory.
2. The cored were laid (along with core wrap and ADL if present) flat at a 30° incline.
3. Front end of the core was placed towards the bottom, so fluids ran downwards towards the front.
4. The insult location was marked at 195 mm from front end of the pad towards the back (MD) and at the middle CD.
5. The flow of the insult was adjusted to be 7 ml/s (85 ml total insulted over 12 seconds)
6. The open end of the insult hose, beaker or funnel was adjusted so it was placed 5 mm from the sample's surface, placed at 90 degrees from the core so the insult ran at 90 degrees vertically towards the insult location.
7. The seconds where first runoff leak was observed was recorded.
8. Weight of total runoff fluid or total weight gain of the core was recorded.

Retentive capacity and incline runoff tests were performed on a diaper in accordance with an exemplary embodiment of the present invention (“C-Fold Unique Core”) and on other diaper constructions, including a diaper with an absorbent system with just a standard core (“SAP Sheet”), and commercially available diapers including “Coterie The Diaper”, “Pampers Pure Protection Diapers” and “Huggies Skin Essentials Diapers”. The results of these tests are shown in Table 2.

TABLE 2

| DTI SKU | C24F-S6-RNB | C24F-S1-RNB | C24J-S1 | C73A-S1 | D16A-S23 |
|---|---|---|---|---|---|
| Date Testing Began | 01/24 | 01/24 | 01/24 | 07/24 | 09/24 |
| Size | 4 | 4 | 4 | 4 | 4 |
| Product Type | Tape | Tape | Tape | Tape | Tape |
| Sample Origin | Prototype | Prototype | Commercial | Commercial | Commercial |
| DIAPER PERFORMANCE | (WFB) C-Fold | (DSF) SAP Sheet | Coterie The | Pampers Pure | Huggles Skin |
| | Unique Core | | Diaper | | Essentials |
| Total Free Swell Capacity (g) | 915 | 799 | 729 | 452 | 553 |
| Centrifugal Retention (g) | 567 | 585 | 569 | 282 | 296 |
| Centrifugal Standard Deviation (g) | 9 | 5 | 29 | 3 | 18 |
| Incline Runoff 1 (g) | 0.00 | 0.00 | 0.00 | 0.70 | 0.00 |
| Incline Runoff 2 (g) | 0.00 | 0.00 | 0.00 | 1.03 | 0.00 |
| Incline Runoff 3 (g) | 0.00 | 0.00 | 0.00 | 1.06 | 0.11 |
| HUT Results | | | | | |
| Leakage Rate | 4.3% | 10.6% | 10.0% | 8.1% | |

Rewet, acquisition time and initial dryness tests were performed on a diaper in accordance with an exemplary embodiment of the present invention in which a channel is not formed in the bottom core (“C-Fold Diaper”), a pant in accordance with an exemplary embodiment of the present invention in which a channel is formed in the bottom core (“C-Fold Pant”) and commercially available diapers including “Coterie The Diaper”, “Coterie The Pant”, “Pampers Pure Protection Diapers” and “Huggies Skin Essentials Diapers”. The results of these tests are shown in Table 3.

TABLE 3

| | | | Coterie | Coterie | | Huggies |
|---|---|---|---|---|---|---|
| | C-Fold | C-Fold | Baby | Baby- | Pampers | Skin |
| Diaper | Diaper | Pant | Diapers | Pants | Pure | Essentials |
| Size | 4 | 4 | 4 | 4 | 4 | 4 |
| Product Weight | 48.9/0.48 | 48.9/0.78 | 38.4/0.85 | 38.2/0.17 | 31.1/0.17 | 34.7/1.74 |
| [g]/SDV | | | | | | |
| Rewet 1 [g] | | | | | | |
| after 140 ml | 0.16 | 0.11 | 0.20 | 0.05 | 0.04 | 6.87 |
| after 210 ml | 0.29 | 0.46 | 0.30 | 0.61 | 0.36 | 19.34 |
| Rewet 2 [g] | | | | | | |
| after 150 ml | 0.15 | 0.10 | 0.25 | 0.04 | 0.01 | 0.36 |
| after 200 ml | 0.16 | 0.30 | 0.18 | 0.57 | 0.24 | 12.03 |
| Acquisition Time/ | | | | | | |
| Rewet 3 | | | | | | |
| 4. Acquisition [s] | 34.5 | 26.3 | 61.6 | 39.3 | 47.1 | 142.7 |
| Rewet after last insult* | 0.99 | 1.56 | 3.82 | 3.06 | 5.82 | 19.12 |
| [g] | | | | | | |
| *Total amount added | 280 | 280 | 280 | 280 | 280 | 280 |
| [ml] | | | | | | |
| Initial Dryness | | | | | | |
| Rewet 140 ml/2 min [g] | 0.3 | 0.8 | 0.7 | 0.6 | 0.1 | 6.9 |

As shown in Tables 2 and 3, the inventive absorbent system including the C-fold core achieves a combination of tripod properties that provides a well-balanced, high-performance product, high in retentive capacity while keeping speed of acquisition fast and rewets low (especially under long term use, focusing on the 2nd and 3rd rewet). Additionally, due to the wicking properties that quickly funnel liquid to the bottom, traditional pulp-plus-SAP core, rewet properties are better showcased in the Initial Dryness test, that uses a funnel instead of a cylinder to administer the insult.

The inventive absorbent system achieves a very quick speed of acquisition, even compared against the market leader (“Pampers”) whose design includes a channeled core, or compared to Coterie The Diaper which has the highest basis weight ADL of any diaper (110 gsm) designed in conjunction with very high speed Sumitomo “grape-shaped” SAP and high pulp to SAP ratio at

In traditional cores, like those made with a mix of SAP and fluff, most of the SAP is located close to the top of the core (near the skin of the user), so that diapers perform quite well in terms of the rewet, being dry to the skin, but very poorly in terms of the acquisition time, with the potential of early leakages.

Similarly, diapers made with SAP sheets or “absorbent paper,” defined as absorbent cores made with no cellulose AKA “pulp-less”, result in similar challenges in achieving competitive speed of acquisition. All tested SAP sheet pulp-less cores result in slow speed of acquisition (example DSF SAP Sheet) or competitive speeds when a lengthwise channel is introduced (example “Coterie The Pant”), however resulting in poor performance in the 30 Degree angle test as saline bleeds through the front end part of the core.

The inventive absorbent system design results in a faster acquisition time. Results are counterintuitive on account of the high loading of SAP, high SAP to pulp ratio and placement of the highest concentration of SAP closest to the user's skin.

Surprisingly, particularly for a diaper with such a high load of SAP close to the skin of the baby, the premade core with the channel resulted in amazing low run offs and the fastest acquisition times. This unexpected result came clear after comparing with other diapers made with a premade core made with SAP (compare to DSG). Additionally, the diaper was tested in a HUT and the test confirmed and validated expectations for extremely low leakages when compared with the market.

In summary, as Table 3 shows, the inventive absorbent system provides the fastest performing diaper in terms of its acquisition time, as well as one of the driest in terms of the rewets, while keeping one of the highest loadings of SAP near the surface.

Further, the channeled core of the inventive absorbent system not only provides very fast acquisition times, but also avoids the need to make size changes in the absorbent core, avoiding the need for core registration and the need for additional downtime and lower efficiencies associated with using interrupted channels.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

We claim:

1. An absorbent system comprising:
a first absorbent core layer, the first absorbent core layer comprising cellulose and superabsorbent material;
a second absorbent core layer disposed above the first absorbent core layer, the second absorbent core layer comprising synthetic material and superabsorbent material, the second absorbent layer having end portions that are folded so that the second absorbent layer has a C-shape;
a first acquisition-distribution layer disposed over the second absorbent core layer;
a core wrap disposed around the first absorbent core layer, the second absorbent core layer, and the first acquisition-distribution layer; and
a second acquisition-distribution layer disposed over the core wrap.

2. The absorbent system of claim 1, wherein the second absorbent core layer comprises a nonwoven layer disposed between two carrier layers.

3. The absorbent system of claim 2, wherein the carrier layers comprise spunbond material.

4. The absorbent system of claim 1, wherein the second absorbent core layer comprises a channel formed between the end portions.

5. The absorbent system of claim 1, wherein the first absorbent core layer comprises a channel.

6. The absorbent system of claim 5, wherein the first absorbent core layer comprises a first portion and a second portion, and the channel is formed by a space between the first portion and the second portion.

7. The absorbent system of claim 5, wherein the channel is formed by a portion of the second absorbent core layer that has a reduced amount of superabsorbent material.

8. The absorbent system of claim 5, wherein the channel is formed by a portion of the second absorbent core layer that has reduced thickness.

9. The absorbent system of claim 1, wherein the first acquisition-distribution layer is larger than the second acquisition-distribution layer.

10. The absorbent system of claim 1, wherein the second acquisition-distribution layer extends less than a full length of the absorbent system.

11. An absorbent article comprising the absorbent system of claim 1.
